(19) United States
(12) Patent Application Publication
(10) Pub. No.: US 2007/0213623 A1
(43) Pub. Date: Sep. 13, 2007

(54) Apparatus and method for vertically positioning a monitoring transducer relative to a patient

(76) Inventors: William Wekell, Bellevue, WA (US); Andrew Nara, Chardon, OH (US)

Correspondence Address: Marcus Simon, Esq.;DORSEY & WHITNEY LLP, Suite 3400, 1420 Fifth Avenue, Seattle, WA, 98101, US

(21) Appl. No.: 11/371,359

(22) Filed: Mar. 7, 2006

(51) Int. Cl. A61B 5/02

(52) U.S. Cl. 600/486

(57) ABSTRACT

The present invention is directed to an apparatus and method for positioning a transducer relative to a patient. In one embodiment, a transducer support having a fluid sensing transducer also includes an illuminator coupled to the support to generate visible radiation and to direct the visible radiation along a first optical axis. A reflective surface receives the visible radiation emitted along the first optical axis and directs the visible radiation along a second optical axis and onto an predetermined elevational position on a patient. In another embodiment, a method includes directing visible radiation in a first direction and onto a reflective surface that reflects the visible radiation in a second direction and towards the patient, projecting the visible radiation onto an external portion of the patient to form an illuminated area on the patient, and aligning the transducer with a predetermined elevation on the surface of a patient.

### TECHNICAL FIELD

The present invention relates generally to patient monitoring devices for medical use. In particular, the invention is an apparatus and method for accurately positioning one or more patient monitoring pressure transducers relative to a patient.

### BACKGROUND OF THE INVENTION

Blood pressure is the most common index of cardiovascular performance presently known. In general, two methods are used to measure and/or monitor blood pressure. A commonly used non-invasive blood pressure measurement method employs a sphygmomanometer to compress an artery and a stethoscope to detect audible characteristics associated with blood flow while the compression of the artery is reduced to allow blood to flow through the artery. In contrast, invasive blood pressure measurement methods generally involve direct intra-corporeal measuring and monitoring of blood pressure.

For critically-ill patients, invasive blood measurement methods are favored for several reasons. First, a blood pressure determination using an invasive method greatly enhances the accuracy of the blood pressure determination, since the measurement is not dependent on sphygmomanometer cuff placement or the detection of an audible characteristic. Additionally, an invasive blood pressure determination allows the blood pressure of the patient to be monitored continuously, as opposed to an intermittent measurement using a non-invasive method. An invasive blood pressure determination also permits the rapid detection of any change in the cardiovascular activity of the patient, which may be critically important in emergency situations. Moreover, invasive blood measurement methods may also be used to monitor the blood pressure at selected internal locations within the body of a patient. For example, it is often advantageous to measure and monitor the blood pressure within the chambers of the heart.

Invasive blood pressure measurement and monitoring generally involves the insertion of a catheter into a selected blood vessel. For example, when it is desired to measure and monitor arterial blood pressure, the catheter is inserted into a radial artery. Correspondingly, if it is desired to measure and monitor venous blood pressure, the catheter may be inserted into the antecubital, radial, tubular or subclavian vein. In any event, the catheter is first filled with a sterile saline solution and de-bubbled. A hypodermic needle is then inserted into the selected blood vessel, and the catheter is then threaded through the hypodermic needle and directed along the blood vessel until the tip of the catheter is positioned at a location where the blood pressure measurement is desired. When the catheter is suitably positioned, the needle may be removed, and the opening may be taped to secure the catheter tip at the selected location. The opposing end of the catheter is coupled to pressure tubing that is also similarly filled with a saline solution. The pressure tubing is then coupled to a pressure transducer capable of detecting pressures transmitted from the selected blood pressure location within the patient. The pressure transducer is, in turn, coupled to an external blood pressure monitoring device and/or other devices, such as a visual display that permits the blood pressure waveform of the patient to be viewed.

The accuracy of an invasive blood pressure determination using the foregoing method depends upon the careful vertical alignment of the pressure transducer with the vertical position of the catheter tip lodged within the patient. If, for example, the pressure transducer is located at a position below the catheter tip, the indicated blood pressure will be higher than the patient's actual blood pressure. Correspondingly, if the pressure transducer is located at a position above the catheter tip, the indicated reading will be lower than the patient's actual blood pressure. Accordingly, careful alignment of the transducer with the vertical position of the catheter tip is a critical concern in blood pressure determinations.

In one prior art method, the pressure transducer is adjustably positioned on a vertical support, and a leveling device such as a carpenter's level is positioned between the patient and the pressure transducer. The position of the transducer on the support is then vertically adjusted so that it is approximately level with a reference mark placed on an external portion of the patient's body. Although the foregoing method is effective, it nevertheless exhibits numerous shortcomings. For example, a variety of equipment is often positioned around the patient that may preclude the use of a generally unwieldy leveling device, such as the carpenter's level. In another prior art method, as disclosed in U.S. Pat. No. 5,280,789 to Potts, a vertical alignment device is disclosed that may be removably attached to a transducer mounting bracket. The device includes a laser light source that projects a coherent beam of light outwardly towards a patient The transducer mounting bracket is then vertically adjusted until a light spot from the laser source is aligned with a reference mark positioned on an exterior portion of the patient. Although the disclosed device constitutes a significant improvement in the state of the art, it discloses the projection of only a single point of light onto the patient, which may be difficult for persons attending the patient to locate in conditions of elevated ambient light and/or conditions where the vertical alignment device is substantially misaligned with the reference mark on the patient when the device is set up. Additionally, the disclosed device does not permit the beam to be positioned independently of the mounting bracket.

In yet another prior art device, as disclosed in U.S. Pat. No. 6,071,243 to MacEachern, another vertical alignment device is disclosed that similarly uses a laser to illuminate a reference mark positioned on a patient. The disclosed device, however, similarly projects a single point of light, and accordingly has many of the shortcomings present in the foregoing prior art device. The disclosed device similarly does not permit the beam to be directed independently relative to the vertical alignment device.

What is needed is a patient monitoring system having a leveling device that may be conveniently aligned with a desired position on a patient so that a pressure transducer may be accurately vertically aligned.

### SUMMARY OF THE INVENTION

The present invention is directed to an apparatus and method for accurately positioning one or more patient monitoring pressure transducers relative to a patient. In one aspect, the apparatus includes a transducer support configured to support at least one fluid sensing transducer, an illuminator coupled to the support to generate visible radiation and to direct the visible radiation along a first optical axis. A reflective surface is positioned adjacent to the illuminator to receive visible radiation emitted along the first optical axis and to direct the visible radiation along the second optical axis and onto an predetermined elevational position on a patient. In another aspect, the apparatus includes a transducer mount supporting at least one transducer, the mount being movable relative to a selected elevational location in the patient, and an illuminator that generates a beam of visible radiation defining an optical path extending from a illumination source to a surface of the patient. A reflector is positioned in the optical path to receive the beam of visible radiation and to direct the beam in a second direction. In still another aspect, a method includes directing visible radiation in a first direction and onto a reflective surface that reflects the visible radiation in a second direction and towards the patient, projecting the visible radiation onto an external portion of the patient to form an illuminated area on the patient, and aligning the transducer with a predetermined elevation on the surface of a patient.

### BRIEF DESCRIPTION OF THE DRAWINGS

FIG. 1 is an isometric view of a patient monitoring system according to an embodiment of the invention.

FIG. 2 is a partial cutaway view of the transducer support showing an illuminator according to another embodiment of the invention.

FIG. 3 is a partial cutaway view of the transducer support showing an illuminator according to still another embodiment of the invention.

FIG. 4(a) through 4(e) are images formed by the embodiment of FIG. 3.

### DETAILED DESCRIPTION OF THE INVENTION

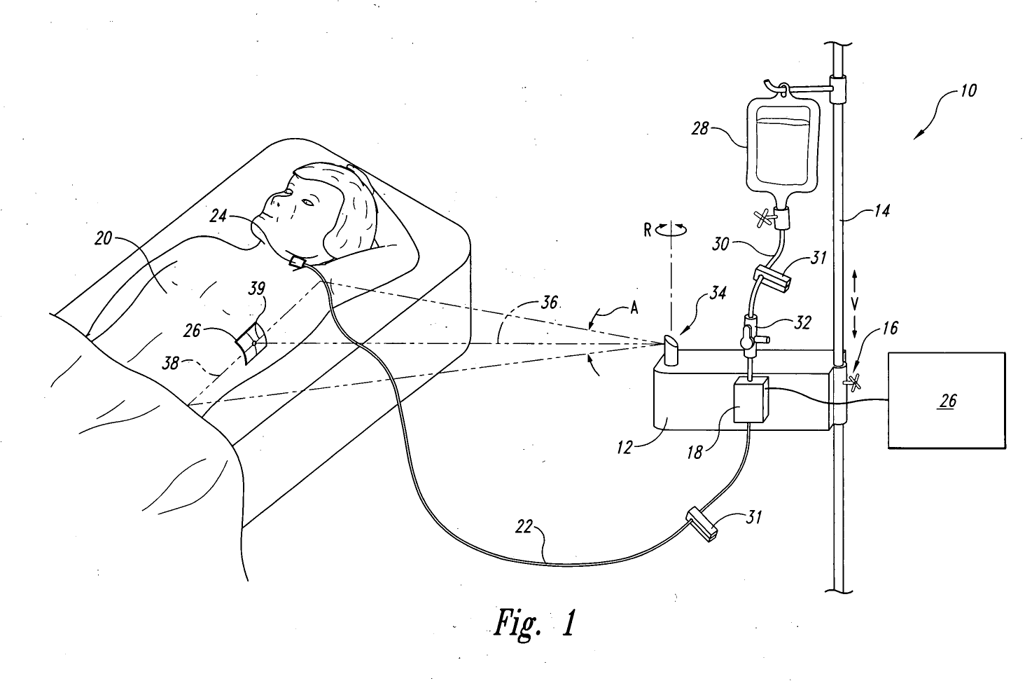

The present invention is generally directed to an apparatus and method for patient monitoring devices for medical use, and more particularly, to an apparatus and method for accurately positioning one or more patient monitoring transducers relative to the patient. Many of the specific details of certain embodiments of the invention are set forth in the following description and in FIGS. 1 through 4 to provide a thorough understanding of such embodiments. One skilled in the art will understand, however, that the present invention may have additional embodiments, or that the present invention may be practiced without several of the details described in the following description.

FIG. 1 is an isometric view of a patient monitoring system 10 according to an embodiment of the invention. The system 10 includes a transducer support 12 configured to be attached to a vertical support 14, such as an IV stand, or other similar vertical support devices. The transducer support 12 is removably attached to the vertical support 14 so that the transducer support 12 may be translated along a length of the vertical support 14 in a direction V and further includes a clamping device 16 to retain the transducer support 12 in a selected position on the vertical support 14. The transducer support 12 is also configured to support a pressure transducer 18 capable of measuring and monitoring the blood pressure of a patient 20. Although the transducer support 12 shows a single transducer 18 mounted thereon, one skilled in the art will readily understand that more than one pressure transducer 18 may be supported by the transducer support 12, so that blood pressure monitoring and measurement may occur simultaneously at more than a single position within the body of the patient 20. The transducer 18 is coupled to a pressure tube 22 that extends from the transducer 18 to a distal end of a catheter 24. The apical tip (not shown) of the catheter 24 is inserted into the patient 20 and extends into the patient 20 to a desired location. A vertical location of the apical tip of the catheter 24 is indicated by a target 26 that may be placed externally on the patient 20. The transducer 18 is further coupled to a saline bag 28 through a saline tube 30 and a flow valve 32 to allow the pressure tube 22 and the catheter 24 to be purged with a saline solution. Line restrictors 31 positioned on the saline tube 30 and the pressure tube 22 may be used to assist in the purging process. The pressure transducer 18 is electrically coupled to a monitoring device 26 configured to process signals received from the pressure transducer 18 and to generate a visual image of the blood pressure level if desired.

Still referring to FIG. 1, the transducer support 12 further includes an illuminator 34 capable of projecting a light beam 36 outwardly from the transducer support 12 and towards the patient 20. In one particular embodiment, the transducer support 12 includes an illuminator 34 that projects a linear beam 36 towards the patient 15 that may further be rotated about an axis R so that the beam 36 may be swept through an angle A. In another particular embodiment, the transducer support 12 includes an illuminator 34 that may include beam forming optics so that a line 38, or an image 39 may be projected onto the patient 15. The foregoing embodiments will be described in greater detail below.

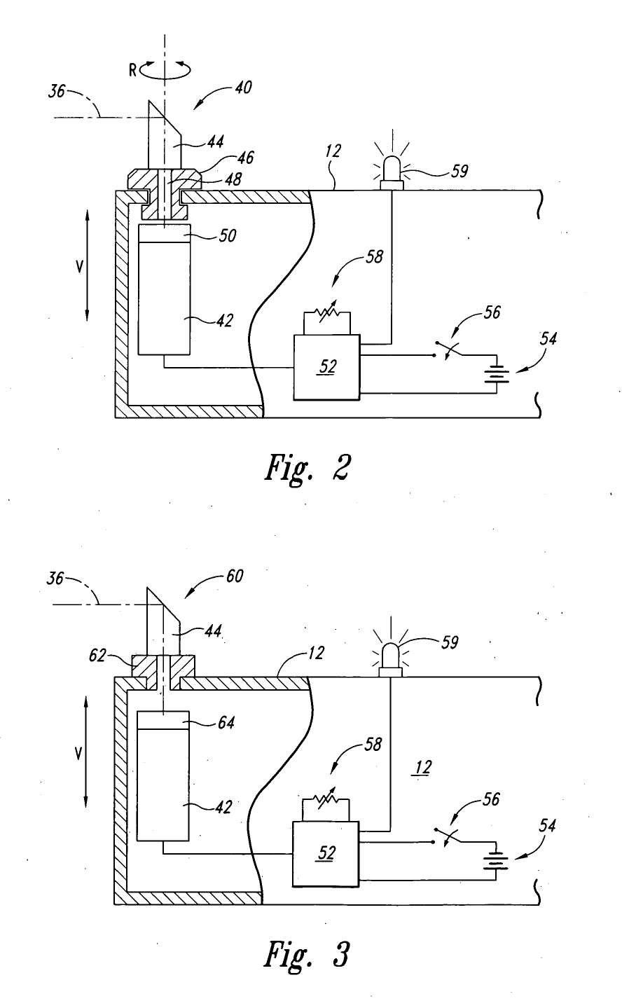

FIG. 2 is a partial cutaway view of the transducer support 12 of FIG. 1 showing an illuminator 40 according to another embodiment of the invention. The illuminator 40 includes an illumination source 42 that is mounted within the transducer support 12 so that the light beam 36 is directed in a vertical direction V and into a reflective prism 44 that reflects the beam 36 in a direction that is approximately perpendicular to the direction V. The reflective prism 44 may include a reflective material disposed on a surface of the prism 44 to reflect the beam 36. Alternately, the prism 44 may be formed so that it includes a surface approximately equal to the critical angle so that the prism 44 becomes internally reflective. In either case, the prism 44 is fixedly positioned on a mount 46 having a centrally disposed aperture 48 that is substantially in alignment with the beam 36. The mount 46 is rotatably coupled to the transducer support 12 so that the prism 44 may be rotated in a direction R so that the beam may be swept through an angle A, as shown in FIG. 1. The mount 46 may be configured so that the rotation of the mount 46 is limited to a rotate through an angle of less than 360 degrees so that the projection of the beam 36 is confined to a predetermined angular range. Alternately, the mount 46 may be configured so that the beam 36 may be continuously rotated through an angle of 360 degrees. Although FIG. 2 shows a prism 44 that reflects the beam 36 towards the patient 20, one skilled in the art will readily recognize that other reflective devices having a reflective surface are well known, and may be used instead of the prism 44.

Still referring to FIG. 2, the illumination source 42 may include an incandescent light source, but preferably includes a coherent light source such as a semiconductor diode laser capable of continuous wave (CW) operation. In one aspect, the diode laser may have a wavelength of about 635 nm. One suitable diode laser is the LD-635-51 diode laser available from Lasermate Group, Inc. of Pomona, Calif. although other alternative diode laser devices exist. The illumination source 42 may also include an optical device 50 that is positioned between the source 42 and the prism 44 to further condition the beam 36. In one aspect, the optical device 50 may comprise a collimating lens coupled to a diode laser. The illumination source 42 may be coupled to a controller 52 that is further coupled to a power source 54 that may be connected to the controller 52 by means of a manually-actuated switch 56. A manually-adjustable potentiometer 58 may also be coupled to the controller 52 that permits the intensity of the beam 36 to be controlled when the illumination source 42 is energized. The controller 52 may also be coupled to a pilot lamp 59 that illuminates when the illumination source 42 is energized, so that the operation of the illuminator 40 is readily apparent.

The foregoing embodiment advantageously permits a beam from the illuminator to be independently directed so that the beam may be swept through a predetermined angular range. Accordingly, the foregoing embodiment allows the beam to be more conveniently directed towards a patient without requiring the vertical support to be moved.

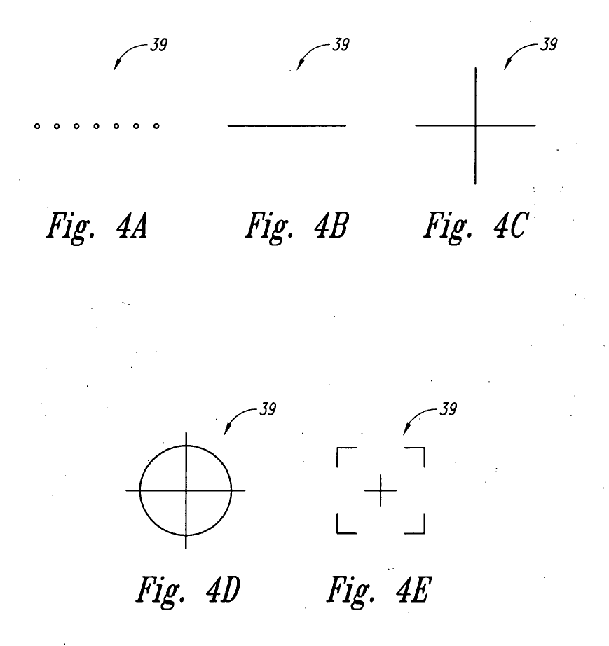

FIG. 3 is a partial cutaway view of the transducer support 12 of FIG. 1 showing an illuminator 60 according to another embodiment of the invention. Many of the details of the present embodiment are discussed in detail in connection with FIG. 2 and in the interest of brevity, will not be discussed further. As in the previous embodiment, the illuminator 60 includes an illumination source 42 that is mounted within the transducer support 12 so that the beam 36 is vertically directed as it emanates from the illumination source 42. The illuminator 60 further includes a prism 44 that may be held in a fixed relationship relative to the support 12 by a mount 62. The illumination source 42 is coupled to an image-generating optical element 64 that generally diffracts the beam 36 generated by the illumination source 42 to produce a pre-selected image 39 when projected onto an external portion of the patient 20 (see FIG. 1). Referring now to FIG. 4, the pre-selected image 39 may include a linear array of dots, as shown in FIG. 4(a) or a line of predetermined length, as shown in FIG. 4(b). Other image-generating optical elements 64 may be employed to produce still other images. For example, an element 64 may be used to produce a cross-hair pattern, as shown in FIGS. 4(c) through (e) when the beam 36 is projected onto an external portion of the patient 15. Referring to FIG. 3, image-generating optical elements 64 suitable for forming the images as shown in FIGS. 4(a) through 4(e) are the L50 Series diffractive pattern generators available from Lasermate Group, Inc. of Pomona, Calif. although other suitable image-generating optical elements exist.

The foregoing embodiment advantageously allows the light projected from the illumination source to be easily detected by projecting an image onto the patient while the device is being leveled. As noted earlier, finding a single light dot under conditions of elevated ambient light may be difficult, particularly in situations where the projected beam in substantially misaligned with the patient.

Although the foregoing has discussed pressure measurement within the specific context of invasive blood pressure measurement, it is understood that the foregoing is also applicable to pressure measurements in other regions of the body. For example, the various embodiments of the present invention may, without significant modification, be used to measure and monitor the intercranial pressure in a patient. Additionally, from the foregoing it will be appreciated that, although specific embodiments of the invention have been described herein for purposes of illustration, various modifications may be made without deviating from the spirit and scope of the invention. For example, certain features shown in the context of one embodiment of the invention may be incorporated in other embodiments as well. Accordingly, the invention is not limited by the foregoing description of embodiments except as by the following claims.

1. An apparatus for positioning a fluid sensing transducer in a patient monitoring system, comprising:
a transducer support configured to support at least one fluid sensing transducer;
an illuminator carried by the support to generate visible radiation and to direct the visible radiation along a first optical axis; and
a reflective surface carried by the support and positioned adjacent to the illuminator to receive visible radiation emitted along the first optical axis and to direct the visible radiation along a second optical axis that is different from the first optical axis and has a vertical position corresponding to the vertical position of the transducer support.

2. The apparatus of claim 1 wherein the first optical axis is approximately perpendicular to the second optical axis.

3. The apparatus of claim 2 wherein the transducer support further comprises a clamping device that is coupled to a fixed support that extends in a direction that is approximately parallel to the first optical axis, the clamping device being adjustably positionable along the fixed support.

4. The apparatus of claim 1 wherein the reflective surface further comprises a reflective prism positioned adjacent to the illuminator.

5. The apparatus of claim 1 wherein the reflective surface is rotatable about an axis approximately parallel to the second optical axis.

6. The apparatus of claim 1 wherein the reflective surface is fixed relative to the second optical axis.

7. The apparatus of claim 1, further comprising a controller coupled to the illuminator that is configured to control at least an intensity of the visible radiation emitted by the illuminator.

8. The apparatus of claim 1 wherein the illuminator further comprises an incandescent light source.

9. The apparatus of claim 1 wherein the illuminator further comprises a coherent light source.

10. The apparatus of claim 9, wherein the illuminator further comprises a semiconductor diode laser light source.

11. The apparatus of claim 9 wherein the first optical axis defines a first optical beam path, and the illuminator further comprises an image-forming diffraction optic positioned in the first optical beam path.

12. The apparatus of claim 9 wherein the first optical axis defines a first optical beam path, and the illuminator further comprises a collimating optic positioned in the first optical beam path.

13. The apparatus of claim 1, wherein the fluid sensing transducer includes a blood pressure sensor.

14. An apparatus for positioning at least one pressure-sensing transducer relative to a patient, comprising:
a transducer mount supporting the at least one transducer, the mount being movable relative to a selected elevational location in the patient; and
an illuminator carried by the transducer mount that is operable to generate a beam of visible radiation defining an optical path, the illuminator having a rotatable element that is configured to rotate the optical path about a vertical axis extending from the transducer mount.

15. The apparatus of claim 14 wherein the illuminator further comprises a reflector to redirect the optical path from a first direction to a second direction that is approximately perpendicular to the first direction.

16. The apparatus of claim 15 wherein the reflector further comprises a reflective prism positioned in the optical path.

17. The apparatus of claim 15 wherein the reflector is rotatable about an axis approximately parallel to the first direction.

18. The apparatus of claim 15 wherein the reflector is fixed relative to the second direction.

19. The apparatus of claim 14 wherein the transducer mount further comprises a clamping device that is fixably attachable to a support.

20. The apparatus of claim 14 further comprising a controller coupled to the illuminator that is configured to control at least an intensity of the visible radiation emitted by the illuminator.

21. The apparatus of claim 14 wherein the illuminator further comprises an incandescent light source.

22. The apparatus of claim 14 wherein the illuminator further comprises a coherent light source.

23. The apparatus of claim 22 wherein the illuminator further comprises a semiconductor diode laser light source.

24. The apparatus of claim 23 wherein the illuminator further comprises an image-forming diffraction optic positioned in the optical path.

25. The apparatus of claim 23 wherein the illuminator further comprises a collimating optic positioned in the optical path.

26. The apparatus of claim 14 wherein the at least one transducer includes a blood pressure sensor.

27. A method for aligning a transducer for measuring a bodily fluid pressure with a predetermined vertical elevation in a patient, comprising:
directing visible radiation in a first direction and onto a reflective surface that reflects the visible radiation in a second direction that extends towards the patient, the second direction being positioned at a height that is at substantially the same height as the transducer;
projecting the visible radiation onto an external portion of the patient to form an illuminated area on the patient; and
using the light projected onto the external portion of the patient to align the transducer with the predetermined elevation.

28. The method of claim 27 wherein directing visible radiation in a first direction and onto a reflective surface that reflects the visible radiation in a second direction further comprises directing coherent visible radiation in the first direction and onto a reflective prism that directs the coherent visible radiation in the second direction.

29. The method of claim 27 wherein directing visible radiation in a first direction and onto a reflective surface that reflects the visible radiation in a second direction further comprises directing the visible radiation in a first direction and reflecting the visible radiation in a second direction that is approximately perpendicular to the first direction.

30. The method of claim 27 wherein projecting the visible radiation onto an external portion of the patient to form an illuminated area on the patient further comprises projecting a coherent beam of visible radiation having a defined beam diameter onto the external portion to form an illuminated spot on the patient having a diameter approximately equal to the beam diameter.

31. The method of claim 27 wherein directing visible radiation in a first direction further comprises directing a coherent beam of visible radiation in the first direction and diffracting the coherent beam with a diffraction optic, and wherein projecting the visible radiation onto an external portion of the patient to form an illuminated area on the patient further comprises projecting an image onto an external portion of the patient.

32. The method of claim 27, wherein aligning the transducer with the predetermined elevation further comprises adjustably coupling at least one transducer to a support that is approximately parallel with the first direction and translating the at least one transducer along the support until the illuminated area approximately coincides with the predetermined elevation.

33. A method for aligning a transducer for measuring a bodily fluid pressure with a predetermined vertical elevation in a patient, comprising:
directing a beam of visible radiation along an optical path that extends substantially horizontally from a location corresponding to the vertical position of the transducer;
rotating the optical path about a substantially vertical axis until the beam of visible radiation intersects the patent; and
adjusting the vertical position of the transducer until the beam of visible radiation intersects the patient at substantially the vertical position of the predetermined elevation.

34. The method of claim 33 wherein directing the beam of visible radiation along an optical path comprises directing the beam of visible radiation in a first direction and onto a reflective surface that reflects the visible radiation in a second direction.

35. The method of claim 33 wherein the visible radiation comprises coherent visible radiation.

36. The method of claim 33, further comprising placing a visible marking on the patient at substantially the vertical position of the predetermined elevation, and wherein the beam of visible radiation intersects the patient with a beam diameter that is approximately equal to a diameter of the visible marking.
